(19) United States
(12) Patent Application Publication
(10) Pub. No.: US 2012/0078256 A1
(43) Pub. Date: Mar. 29, 2012

(54) MEDICAL DRILL

(75) Inventors: Christopf HERDRICH, Wyhl am Kaiserstuhl (DE); Eberhard KÕRNER, Knittlingen (DE); Peter BERG, Kraichtal (DE)

(73) Assignee: RICHARD WOLF GMBH, Knittlingen (DE)

(21) Appl. No.: 13/235,804

(22) Filed: Sep. 19, 2011

(30) Foreign Application Priority Data
Sep. 23, 2010 (DE) 10 2010 046 419.8

(51) Int. Cl. A61B 17/16

(52) U.S. Cl. 606/80

(57) ABSTRACT

A medical drill includes a shank and a cutting body arranged in a stationary manner in a region of a distal end of the shank. The cutting body projects radially on the shank and includes a cutter facing a proximal end of the shank.

### BACKGROUND OF THE INVENTION

The present invention relates generally to a medical drill.

Operational techniques, with which artificial bone channels which at an end region have a greater cross section compared to the remaining channel need to be drilled, are common in orthopaedics. Such bone channels are necessary with different operation methods for the reconstruction of ligaments. Here, the widened region of the bone channel serves for receiving one end of a ligament transplant which by way of a thread which is fastened on the transplant and which is led through the narrower section of the bone channel, is fixed outside the channel on the bone.

The bore of stepped artificial bone channels, in particular with a reconstruction of the front cruciate ligament, has been found to be problematic, since in this case, the widened regions of the bone channels necessary for fastening the transplant need to be formed on the shinbone and femur at the joint gap of the femorotibial joint.

For this purpose, a system known from EP 1 785 103 A1, serves for the retrograde drilling of bones. Here, a bone channel running out into the femorotibial joint is firstly drilled with a drill, and an additional channel running out in the joint gap of the femorotibial joint is created, via which a drill attachment is attached at the drill end located in the joint gap. Thereafter, with a rearward movement of the drill out of the bore, the widened region of the bone channel which borders the femorotibial joint is drilled open with the drill attachment. Subsequently, the drill attachment must be detached from the drill and removed from the joint gap of the femorotibial joint.

A drill which is known from EP 2 098 177 A1 and which at its distal end includes an additional cutter which may be folded radially out of the drill shank, permits a less complicated procedural manner. With this drill, firstly, with the cutter folded into the shank, a bone channel running out in the femorotibial joint is created, and the region of this channel which is adjacent to the joint is widened with the folded-out additional cutter by way of a rearwardly directed drill movement of the drill. The drill on account of the movable additional cutter has comparatively complicated mechanics for folding the additional cutter in and out, and has a relatively low stability.

### BRIEF SUMMARY OF THE INVENTION

Against this background, it is an objective of a preferred embodiment of the present invention to provide a medical drill which, given a simple construction and simple handling, permits the creation of stepped bores, in particular with the reconstruction of the front cruciate ligament.

The above objective is achieved by a medical drill with a shank and with a cutting body arranged in a stationary manner in a region of a distal end of the shank, projects radially on the shank and includes a cutter facing a proximal end of the shank. Advantageous further designs of this drill are to be deduced from the subsequent description as well as the drawings. Hereby, according to a preferred embodiment of the present invention, the features specified in the claims, in each case per se as well as in a technologically meaningful combination, may further form the solution according to the present invention.

The drill according to a preferred embodiment of the present invention includes a shank on which, in the region of its distal end, a cutting body projecting radially beyond the outer periphery of the shaft is arranged. The arrangement of the cutting body on the shank is a stationary one, so that the cutting body with the shank forms a rigid unit. For example, the shank may not be folded in. The cutting body at its projecting region comprises a cutter which faces the proximal end of the shank. For example, the cutter or cutting edge of the cutting body which projects radially on the outer periphery of the shank points in the direction of the proximal drill end which may be chucked into the drilling machine.

With this comparatively simple design, the medical drill according to a preferred embodiment of the present invention permits the rapid and simple creation of stepped bone channels with a retrograde widening of the bone channels at the bone side which is away from the drill attachment, as are necessary with many operation techniques in the field of orthopaedics. For this, the drill is not driven in rotation for introduction into the part of the bone channel with a smaller diameter, for example, is driven through the bone without rotation of the drill similarly to the application of a chisel, until the distal end region of the drill with the cutting body which is arranged thereon, projects out of the bone at the opposite side of this. Subsequently, the drill is moved back amid the normal drill rotation and in this manner, a widened region of the bone channel to be created is drilled out in a retrograde manner by way of the cutting body whose cutter then faces the bone.

Usefully, one may carry out a pre-drilling through the bone with a normal cylindrical drill, whose diameter corresponds to the smaller diameter of the bone channel to be created in its non-widened section, before breaking through the bone with the drill according to a preferred embodiment of the present invention. The breaking-through of the bone is simplified by way of this, since bone tissue needs only to be removed by the cutting body projecting radially with respect to the drill shank, amid the formation of a groove. Inasmuch as this is concerned, the drill according to a preferred embodiment of the present invention quasi forms a broaching tool, and the cutting body an additional cutter for broaching the groove. After drilling out the widened section of the bone channel, the drill according to a preferred embodiment of the present invention is usefully rotated such that the cutting body is aligned to the clearance profile of the groove and is pulled out of the bone channel in this position, wherein the cutting body moves through the groove.

Preferably, one envisages the cutting body not being directly adjacent the distal end of the drill, but being distanced to this end, so that an end region of the shank projects on the distal side of the cutting body. At its distal end, the shank preferably has an atraumatic rounding. This, with the presence of a starting bore, simplifies the positioning of the drill in the starting bore and permits a simpler centering of the drill in this starting bore.

One advantageous measure for simplifying the leading of the drill through the bone lies in a radial outer side of the cutting body tapering in a preferably wedge-like manner in the distal direction. Accordingly, the side surfaces of the cutting body which delimit the radial outer side of the cutting body may be aligned in a manner such that departing from the proximal-side end of the cutting body, on which its cutter is formed, they taper to one another in the distal direction at least where they are adjacent to the radial side of the cutting body, so that the radially outer side of the cutting body has the shape of an acute triangle. Moreover, a distal end-side of the cutting body which forms a cutter for broaching the groove, may taper outwards in the distal direction in the shape of a triangle.

Advantageously, a longitudinal marking may be formed on the shank in the axis direction of the shank and in the extension of the cutting body. With regard to this marking, it may be the case for example of a straight longitudinal notching on the shank or of a colored longitudinal line deposited on the shank. The rotational position of the cutting body may also be recognized by the user of the drill according to a preferred embodiment of the present invention, even if this is not visible to the user, for example is located in the body interior in a joint gap, on account of the longitudinal marking formed on the shank. This is advantageous inasmuch as the drill, after a retrograde stepped drilling, may only be removed from the bone again when the cutting body is aligned to the clearance profile of the groove produced by the cutting body. Inasmuch as this is concerned, it is only the position of the cutting body with the initial mortising of the drill into the bone which needs to be known to the user. Usefully, the longitudinal marking extends parallel to the longitudinal axis of the shank at the same angular position as the cutting body. Thus, the user for retracting the drill needs only to bring the longitudinal marking into a position lying opposite the groove, wherein the cutting body automatically aligns with the groove.

Further advantageously, a longitudinal scale running in the longitudinal direction of the shank may be formed on the shank on the proximal side of the cutting body. Accordingly, usefully several markings maybe be formed behind one another at equal distances on the shank in a region between the proximal end of this and the cutting body, and these markings, for example, run transversely to the longitudinal extension of the shank around its periphery. This scale or these markings advantageously permit the determining of the depth of the produced stepped bore with the drill.

A recess for receiving the cutting body may be formed on the drill in the region of the distal end of the shank. This recess is preferably designed in a groove-like manner, wherein the shape and dimensions of the recess correspond to the geometry of the region of the cutting body which to be is introduced into the recess. Advantageously, the cutting body may be releasably fastened in the recess, so that the cutting body may be exchanged when required. This design permits the drill according a preferred embodiment of the present invention to be applied for different drilling tasks as the case may be, with the selection of suitable cutting bodies or by way of their exchange. Moreover, a replacement of the cutting body is possible given any wear. Preferably however, one envisages the drill according to a preferred embodiment of the present invention being design as one piece, for example, the cutting body being an integral constituent of the drill.

As has already been noted, before the application of the drill according to a preferred embodiment of the present invention, it is usefully to carry out a starting bore whose diameter corresponds to the diameter of the bone channel to be created, in its region which is not widened. Advantageously, the outer diameter of the shank corresponds essentially to the inner diameter of this starting bore to be carried out before the application of the drill, or is slightly smaller. In this manner, the shank of the drill according to a preferred embodiment of the present invention, with the creation of the widened region of the bone channel, is led in the starting bore with a little play in the manner of a shaft guidance, which permits an exact alignment and design of the widened region of the bone channel.

Further preferably, one envisages the cutting body projecting laterally on the shank. Accordingly, the cutting body and its cutter project preferably at least 0.5 mm in the radial direction with respect to the peripheral surface of the shank, so that one may create stepped bores, with which the widened section of the bone channel has a diameter which is at least 1 mm larger than the remaining section of the bone channel.

The drill according to a preferred embodiment of the present invention is usefully driven by a drilling machine. The drill at its proximal end advantageously includes a receiver for fastening the drill on the drilling machine. This receiver connects proximally to the shank, wherein preferably a component forms the shank as well as the receiver.

The medical drill according to a preferred embodiment of the present invention may basically be applied whenever stepped bores are to be formed. However, it is particularly advantageous for forming a stepped bore in a bone and in particular for a reconstruction of the front cruciate ligament. With such a reconstruction, such bone channels for fastening a cruciate ligament transplant which in each case have a widened diameter in a section adjacent the joint gap of the femorotibial joint, may be created in a quicker and simpler manner on the shinbone and femur with the drill according to the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings an embodiment which is presently preferred. It should be understood, however, that the invention is not limited to the precise arrangements and instrumentalities shown. In the drawings:

FIG. 1 is a perspective lateral view of a medical drill in accordance with a preferred embodiment of the present invention;

FIG. 2 is an enlarged perspective representation of a distal end of the drill of FIG. 1; and

FIG. 3 is a greatly simplified schematic representation of a bone channel to be created with the drill of FIG. 1.

### DETAILED DESCRIPTION OF THE INVENTION

Certain terminology is used in the following description for convenience only and is not limiting. The words “proximal” and “distal” designate directions in the drawings to which reference is made. The word “outwardly” refers to a direction away from the geometric center of the device, and designated parts thereof, in accordance with the present invention. Unless specifically set forth herein, the terms “a,” “an” and “the” are not limited to one element, but instead should be read as meaning “at least one.” The terminology includes the words noted above, derivatives thereof and words of similar import.

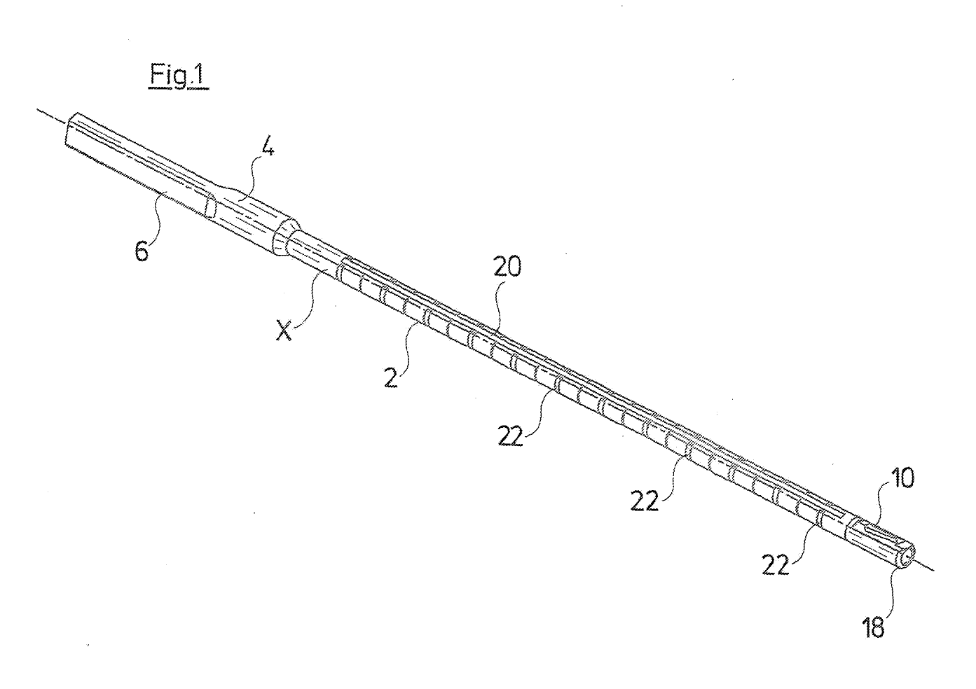

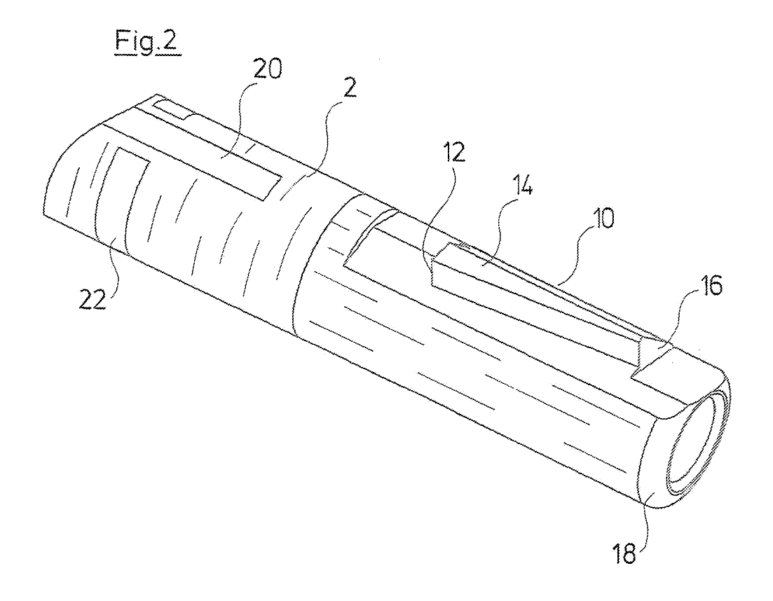

Referring to the drawings in detail, wherein like numerals indicate like elements throughout the several views, FIGS. 1 and 2 show a medical drill preferably including a cylindrical shank 2. On the proximal side, a receiver 4 for clamping the drill in a clamping chuck of a drilling machine preferably connects to the shank 2.

A cutter 12 is preferably formed on an end of the cutting body 10 which points in the direction of the proximal end of the drill. A radial outer side 14 of the cutting body 10 preferably tapers in the distal direction in a wedge-like manner. One distal end-side 16 of the cutting body 10 which is arranged perpendicularly to the longitudinal axis X of the shank 2 tapers in the radial direction in the form of a triangle.

Preferably, the cutting body 10 is not arranged directly on the distal end of the drill, so that a region of the shank 2 projects on the distal side of the cutting body 10. This region of the shank 2 at the end-side is provided with a radius 18 and thus forms an atraumatic tip of the drill.

The shank 2 is preferably provided with a longitudinal marking 20 in a region between the cutting body 10 and the receiver 4 adjacent the shank 2. With regard to the longitudinal marking 20 it is the case of a straight, colored line and/or a recess, which is arranged and directed in the extension of the cutting body 10 and runs parallel to the longitudinal axis X. The longitudinal marking 20 serves for determining the angular position of the cutting body 10 during the application of the drill in the body interior.

Moreover, a longitudinal scale is formed on the shank 2 of the drill in a region on the distal side of the cutting body 10. This longitudinal scale preferably consists of several markings 22 which run in the peripheral direction of the shank 2 and which are arranged one after the other at equal distances in the longitudinal direction of the shank 2. The longitude scale permits the determining of the drill depth.

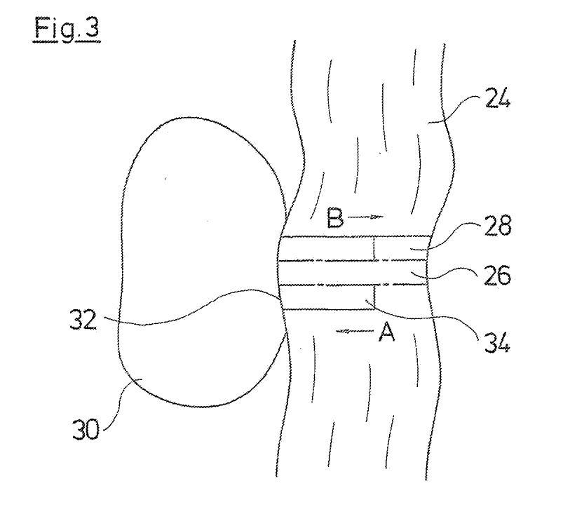

The preferred procedural manner on drilling a stepped bore (FIG. 3) with the represented drill is as follows:

Firstly, the drill is preferably pushed into the bone 24 with a purely linear movement, for example, without drill rotation. Usefully, prior to this, a starting bore 26 leading through the bone 24 (shown dashed in FIG. 3) and whose diameter corresponds to the non-widened region of the bone channel to be created and to the outer diameter of the shank 2, may be created in order to simplify this procedure. The tip formed at the distal end of the drill simplifies the application of the drill in the starting bore 26. Hereby, one usefully takes care that the cutting body 10 or the longitudinal marking 20 is located in a noticeable position, for example 12 o'clock position.

On pushing through the starting bore 26 on the bone 24 in a straight-lined manner, the end-side 16 of the cutting body 10 preferably forms a cutter, with which a longitudinal groove 28 is cut out by way of broaching adjacent a longitudinal groove 28. After pushing through the bone 24 when the cutting body 10 is located outside the bone 24, for example in a cavity 30 located therebehind, the drill is preferably driven in rotation and pulled back in the direction B against the previous pushing direction A. Hereby, the cutter 12 formed on the cutting body 10 comes into contact with the edge 32 of the starting bore 26, and a widened region 34 of the bone channel to be created is cut out with the cutter 12. Hereby, the user of the drill may monitor the drilling depth on drilling out the widened region 34, by way of the longitudinal scale formed on the shank 2. The drive of the drill is switched off when the drilling depth is sufficient. The drill may now be moved manually into a position, in which the cutting body 10 or the longitudinal marking 20 is located in a position which corresponds to that before pushing through the starting bore 26. In this case, it is ensured that the radially projecting cutting body 10 is located within the clearance profile of the longitudinal groove 28 which is adjacent the starting bore 26, so that the drill may be retracted out of the starting bore 26 without any problems. The drilling procedure is then preferably completed.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A medical drill comprising a shank (2) and cutting body (10) arranged in a stationary manner in a region of a distal end of the shank (2), the cutting body projecting radially on the shank (2) and comprising a cutter (12) facing a proximal end of the shank (2).

2. The medical drill according to claim 1, wherein the distal end of the shank (2) tapers to a tip (16) on a distal side of the cutting body (10).

3. The medical drill according to claim 1, wherein a radial outer side (14) of the cutting body (10) tapers in a distal direction in a wedge-like manner.

4. The medical drill according to claim 1, wherein a longitudinal marking (20) is formed on the shank (2) in an axis direction of the shank (2) and in an extension of the cutting body (10).

5. The medical drill according to claim 1, wherein a longitudinal scale (22) running in a longitudinal direction of the shank (2) is formed on the shank (2) on a proximal side of the cutting body (10).

6. The medical drill according to claim 1, wherein an outer diameter of the shank (2) corresponds to an inner diameter of a starting bore (26) created before the application of the drill.

7. The medical drill according to claim 1, wherein the proximal end of the shank (2) comprises a receiver (4) for a drilling machine.

8. The medical drill according to claim 1, wherein the drill forms a stepped bore in a bone (24) for a cruciate ligament reconstruction.
